(19) United States
(12) Patent Application Publication
(10) Pub. No.: US 2014/0046133 A1
(43) Pub. Date: Feb. 13, 2014

(54) SELF-PROPELLING DEVICE

(71) Applicant: FUJIFILM CORPORATION, Tokyo (JP)

(72) Inventors: Takayuki NAKAMURA, Ashigarakami-gun (JP); Tsuyoshi ASHIDA, Ashigarakami-gun (JP); Shinichi YAMAKAWA, Ashigarakami-gun (JP); Yasunori OHTA, Ashigarakami-gun (JP); Charles Alan BRANTINGHAM, St. Paul, MN (US)

(73) Assignee: FUJIFILM CORPORATION, Tokyo (JP)

(21) Appl. No.: 14/035,530

(22) Filed: Sep. 24, 2013

Related U.S. Application Data
(63) Continuation of application No. PCT/JP2012/055044, filed on Feb. 29, 2012.
(60) Provisional application No. 61/467,669, filed on Mar. 25, 2011.

(51) Int. Cl. A61B 1/00

(52) U.S. Cl. CPC A61B 1/00156; A61B 1/0016
USPC 600/114

(57) ABSTRACT

A self-propelling device includes a drive unit and a toroid unit supported thereabout. The toroid unit includes a track structure of a toroid shape and a roller support sleeve inside the track structure. Three belt portions are provided in the track structure with high rigidity. The drive unit includes an inner sleeve for mounting on an introducer of an endoscope, and an outer sleeve supported around the inner sleeve. An inner surface of the roller support sleeve and an outer surface of the outer sleeve extend triangularly. Drive wheels are disposed upstream and downstream of respectively flat portions of the outer sleeve. Two follower rollers are disposed on respectively each of flat portions of the roller support sleeve. The drive wheels and follower rollers nip the belt portions of the track structure to move the track structure endlessly.

### BACKGROUND OF THE INVENTION

1. Field of the Invention

The present invention relates to a self-propelling device secured to an introducer of an endoscope for propulsion and return of the endoscope entered in a gastrointestinal tract, such as a large intestine.

2. Description Related to the Prior Art

In an endoscopic examination, entry of an endoscope into a large intestine is very difficult, because the large intestine is structurally tortuous in the body, and has portions not attached to the inside of a body cavity. Learning its manipulation for entry into the large intestine requires much experience. If the manipulation for entry is technically poor, acute pain may be given to a patient.

A so-called sigmoid colon and transverse colon are body parts where it is said that entry of the endoscope is specially difficult in the large intestine. This is because the sigmoid colon and the transverse colon are not attached in the body cavity unlike other body parts, carry out free changes in the shapes in the range of their length, and deform in the body cavity by the force of contact at the time of entry of the endoscope. Various types of manipulations have been suggested to enable straightening the sigmoid colon and the transverse colon to reduce the contact with a gastrointestinal tract at the time of the entry of the endoscope. Also, a self-propelling device (also referred to as entry aid device for the endoscope) is known (for example, see U.S. Pat. Nos. 6,971,990 and 7,736,300 (corresponding to JP-A 2009-513250)) for propulsion of the endoscope in the gastrointestinal tract to facilitate the entry even for a person unskilled in the manipulation for entry.

The self-propelling device includes a housing, a track structure and a driver. The housing contains an introducer of the endoscope. The track structure is in a toroid form (doughnut form) secured to surround the periphery of the housing about an advancing direction of the endoscope as a central axis. The driver endlessly turns the track structure like a caterpillar to propel the endoscope in the advancing direction or return the endoscope. The track structure is formed from flexible material, and contacts an inner wall of the gastrointestinal tract to exert force for propulsion. The track structure receives force from the inner wall in such manners that a twist may occur in a peripheral direction or that excessive pull may occur in the advancing direction. The track structure should structurally have high rigidity and durability. Especially, portions of the track structure nipped by the drive wheel and the follower roller are likely to degrade because tightly contacted for movement. Low durability of those portions is serious.

### SUMMARY OF THE INVENTION

In view of the foregoing problems, an object of the present invention is to provide a self-propelling device in which twist is prevented in movement of the track structure, and strength of a portion nipped by a drive wheel and follower roller can be increased.

In order to achieve the above and other objects and advantages of this invention, a self-propelling device has a toroid unit, including a track structure shaped in a toroid form with an inner space and movable in circulation, a highly rigid belt portion formed with the track structure to extend in a direction of the circulation, a roller support sleeve, disposed in the inner space of the track structure, and having an inner surface including plural first flat portions and plural first corner portions arranged alternately, and a follower roller, secured to each of the first flat portions, for pressing the belt portion on an inner surface of the track structure. A drive unit includes an inner sleeve for mounting on an introducer of an endoscope, an outer sleeve having an outer surface including plural second flat portions and plural second corner portions arranged alternately, the plural second flat portions being supported by the inner sleeve, covering an outer surface of the inner sleeve, and being opposed to respectively the first flat portions of the roller support sleeve, the plural second corner portions being opposed to respectively the first corner portions of the roller support sleeve, and a drive wheel for nipping the belt portion with the follower roller of the toroid unit, and moving the track structure in circulation.

The track structure includes at least one main sheet shaped in the toroid form, and the belt portion includes at least one reinforcement sheet attached to the main sheet.

The reinforcement sheet is a mesh sheet of resin.

The first flat portions are three first flat portions, the second flat portions are three second flat portions, the first corner portions are three first corner portions, the second corner portions are three second corner portions, the first and second corner portions are arcuate, and thus an inner surface of the roller support sleeve and an outer surface of the outer sleeve extend substantially triangularly.

A passage space is defined between the roller support sleeve and the outer sleeve for moving the track structure, and each of the follower roller and the drive wheel is disposed partially to project into the passage space.

The drive unit includes front and rear lid portions for coaxially supporting the inner sleeve and the outer sleeve.

The front lid portion is formed together with the outer sleeve.

The rear lid portion is formed together with the inner sleeve.

The drive unit has a driver for applying rotational force to the drive wheel.

The driver includes a worm gear for engagement with the drive wheel, and a drive gear for rotating the worm gear.

The drive gear is disposed inside one of the second corner portions of the outer sleeve.

Rotational force is applied to the drive gear with a torque wire by a force source disposed externally of the endoscope.

In the self-propelling device according to the present invention, the track structure does not twist during the movement owing to the belt portion with the increased rigidity. Its resistance to abrasion in relation to the drive wheel and follower roller can be increased, with high durability.

### BRIEF DESCRIPTION OF THE DRAWINGS

The above objects and advantages of the present invention will become more apparent from the following detailed description when read in connection with the accompanying drawings, in which:

FIG. 1 is a schematic view illustrating an endoscope on which a self-propelling device according to the present invention is mounted;

FIG. 2 is a perspective view illustrating appearance of the self-propelling device;

FIG. 3 is a schematic view illustrating a sectional structure perpendicular to an advancing direction of the self-propelling device;

FIG. 4 is an exploded perspective view illustrating a structure of the self-propelling device;

FIG. 5 is a schematic view illustrating a sectional structure in the advancing direction of the self-propelling device;

FIG. 6 is a perspective view illustrating a material sheet for a track structure before forming a bag arrangement of a torpid form;

FIG. 7 is a perspective view illustrating the material sheet for the track structure in a developed form;

FIG. 8 is a section illustrating the material sheet for the track structure partially enlarged;

FIG. 9 is an explanatory view illustrating the material sheet for the track structure formed tubularly;

FIG. 10 is an exploded perspective view illustrating another preferred self-propelling device of a structure of forming front and rear lid portions together with respectively an outer sleeve and an inner sleeve;

FIG. 11 is a schematic view illustrating a sectional structure of the self-propelling device.

### DETAILED DESCRIPTION OF THE PREFERRED EMBODIMENT(S) OF THE PRESENT INVENTION

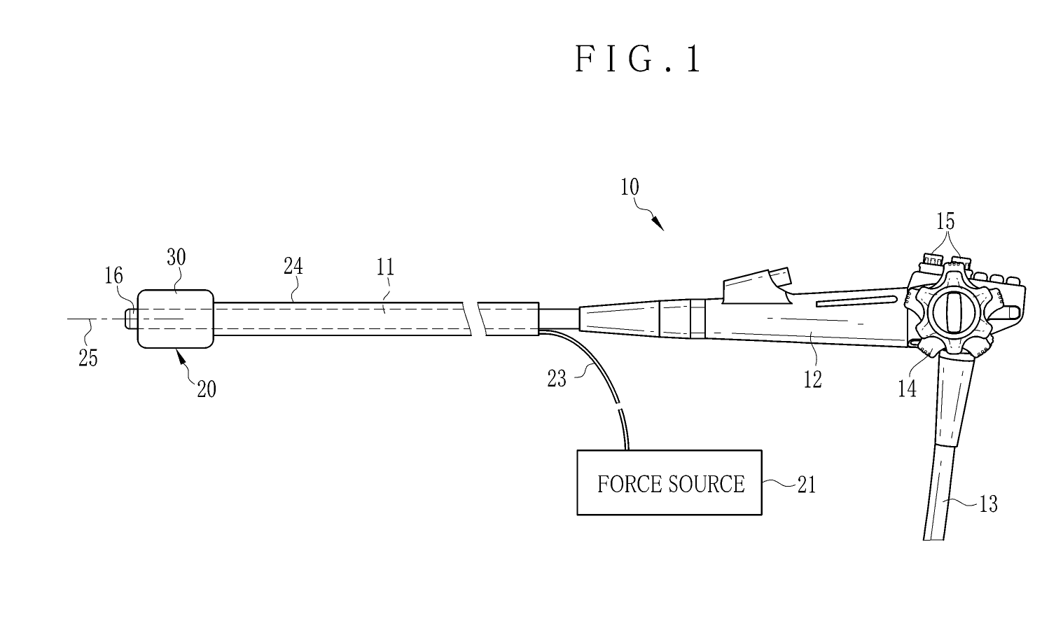

As illustrated in FIG. 1, an endoscope 10 is an electronic endoscope in which a microminiature solid state imaging device (CCD sensor, CMOS sensor and the like) is contained in a tip of a scope. The endoscope 10 includes an introducer 11 (elongated tube), a handle 12 and a universal cord 13. The introducer 11 is entered in a gastrointestinal tract, for example, large intestine. The handle 12 is used for grasping the endoscope 10 and manipulating the introducer 11. The universal cord 13 connects the handle 12 to a processing apparatus and lighting apparatus (not shown). The universal cord 13 includes an air/water supply channel, an output cable for an imaging signal, and a light guide. Also, the handle 12 has angle adjusting knobs 14 (steering wheels) and a control button 15.

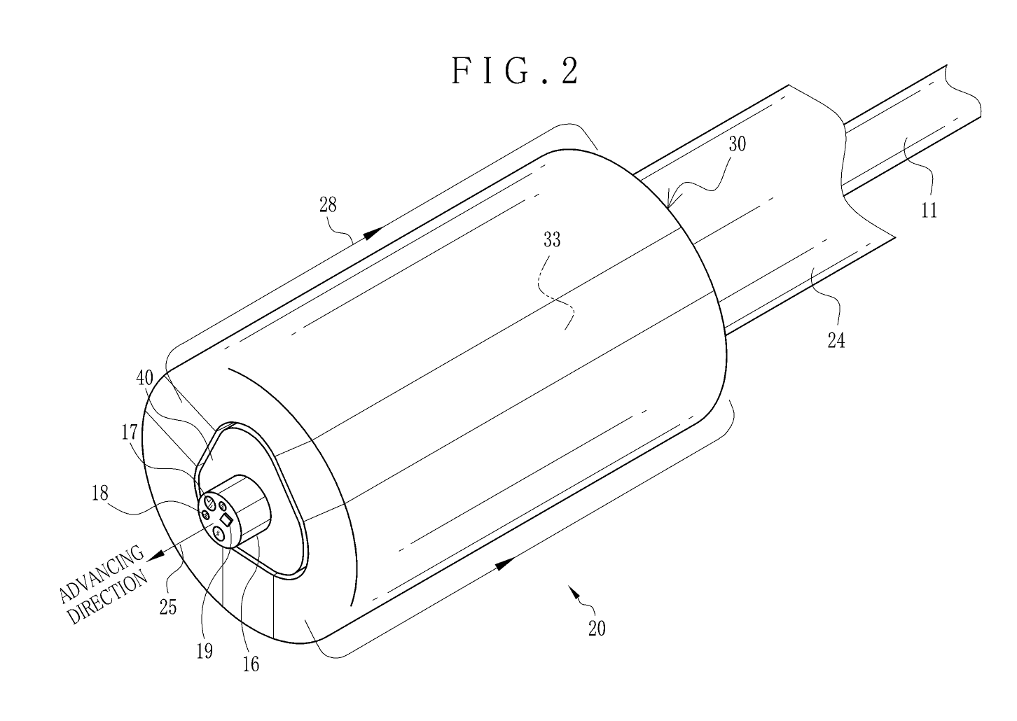

The angle adjusting knobs 14 are rotated at the time of adjusting a steering direction and steering amount of the introducer 11. The control button 15 is used for supply of air/water, suction and various functions. The introducer 11 is a bar-shaped device with flexibility. A tip device 16 has a viewing window 17, lighting windows 18, an air/water supply nozzle 19 and the like (See FIG. 2). A self-propelling device 20 (propulsion assembly) is secured to the tip device 16. The self-propelling device 20 operates to move the introducer 11 forwards or backwards in a gastrointestinal tract.

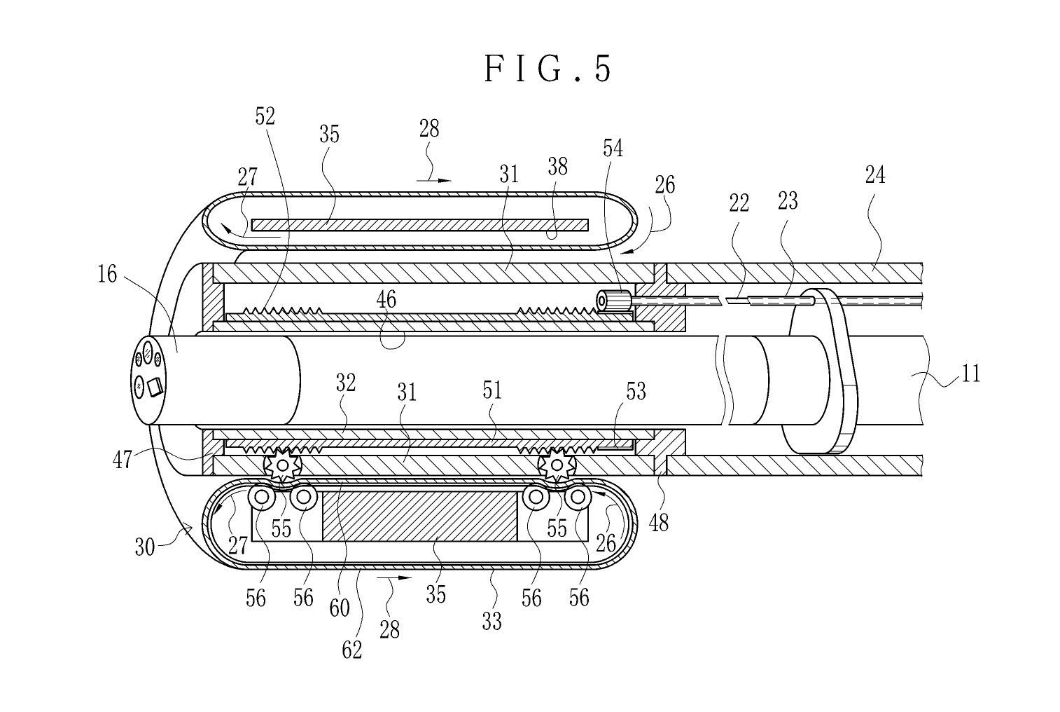

A force source 21 (motor unit) drives the self-propelling device 20. A torque wire 22 (See FIG. 5) is connected with the force source 21 for transmitting rotational torque to move the self-propelling device 20. Nearly the entirety of the torque wire 22 is penetrated through a protection sheath 23. The torque wire 22 is rotated inside the protection sheath 23 by driving of the force source 21. As is not shown, the force source 21 is connected to an input unit, which includes buttons and a speed changing button. The buttons are for inputting instructions for forward movement, backward movement and stop of the self-propelling device 20. The speed changing button is for changing a moving speed of the self-propelling device 20.

An overtube 24 is externally fitted on nearly the entirety of the introducer 11. The protection sheath 23 is contained between the overtube 24 and the introducer 11. The overtube 24 is in a bellows structure compressible and expandable along a longitudinal axis 25.

As illustrated in FIG. 2, the self-propelling device 20 has a drive unit 40 which is entered in a central space in a track structure 30 (endless track device) formed in a bag arrangement of a toroid form (doughnut form). A return run 60 or lower run of the track structure 30 contacts the drive unit 40 and is pushed out along the longitudinal axis 25 of the endoscope 10. A working run 62 or upper run of the track structure 30 in contact with an inner wall of the gastrointestinal tract is moved in a direction of an arrow 28 opposite to an advancing direction, so that the entirety of the track structure 30 is moved endlessly. Thus, the endoscope 10 moves forward relative to the inner wall of the gastrointestinal tract. The track structure 30 is formed from material with flexibility, and compressibility/expandability, for example, biocompatible plastic material or rubber.

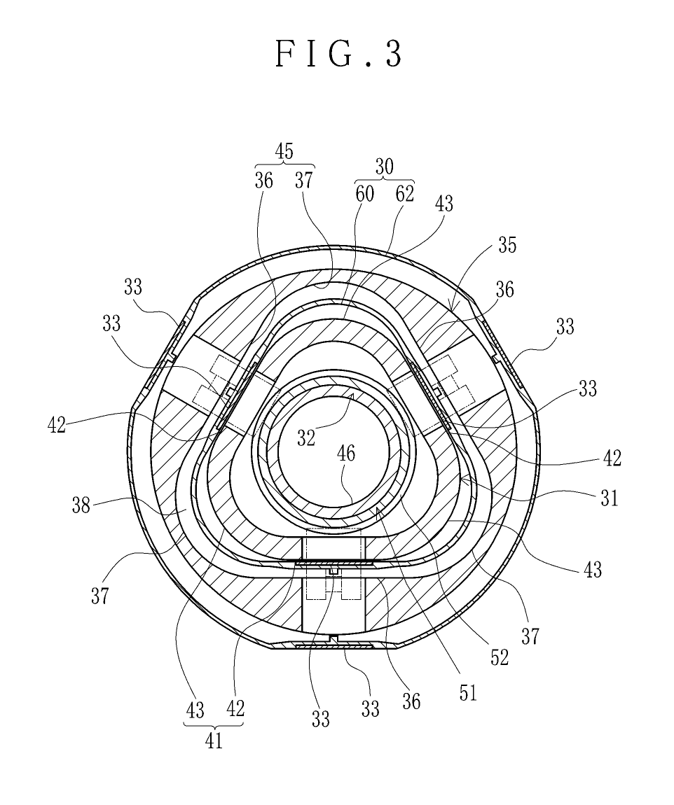

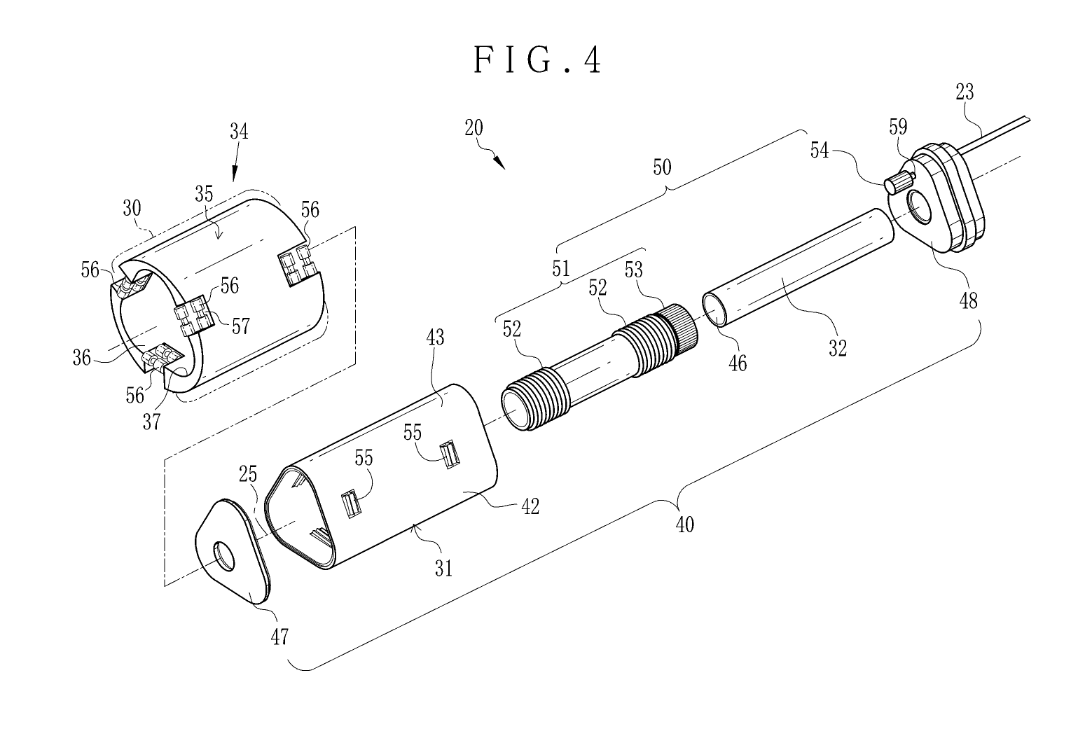

As illustrated in FIGS. 3-5, the self-propelling device 20 includes a toroid unit 34 (barrel unit) and the drive unit 40. The toroid unit 34 is constituted by the track structure 30 and a roller support sleeve 35 (barrel sleeve). The track structure 30 has a belt portion 33 with high rigidity extending in the advancing direction of the above-described introducer, and formed in a bag arrangement of a toroid form. In the roller support sleeve 35 is defined an inner surface 45. The inner surface 45 includes three (first) flat portions 36 and three (first) corner portions 37. Follower rollers 56 are secured to respectively the flat portions 36, and press the belt portion 33 inside the track structure 30. The corner portions 37 are positioned between adjacent ones of the three flat portions 36, and have curved surfaces with a predetermined curvature. A groove 57 is formed at the center of the follower rollers 56.

The drive unit 40 is constituted by an inner sleeve 32 (shaft sleeve) and an outer sleeve 31 (support sleeve). The inner sleeve 32 has a receiving hole 46 in which the tip device 16 is mounted removably. The outer sleeve 31 is supported by the inner sleeve 32 and covers its outer surface. An outer surface 41 is defined on the outer sleeve 31 triangularly, and is constituted by three (second) flat portions 42 and three (second) corner portions 43. The flat portions 42 are disposed to face the flat portions 36 of the toroid unit 34. The corner portions 43 are positioned to face the corner portions 37 of the toroid unit 34, and have curved surfaces with a predetermined curvature.

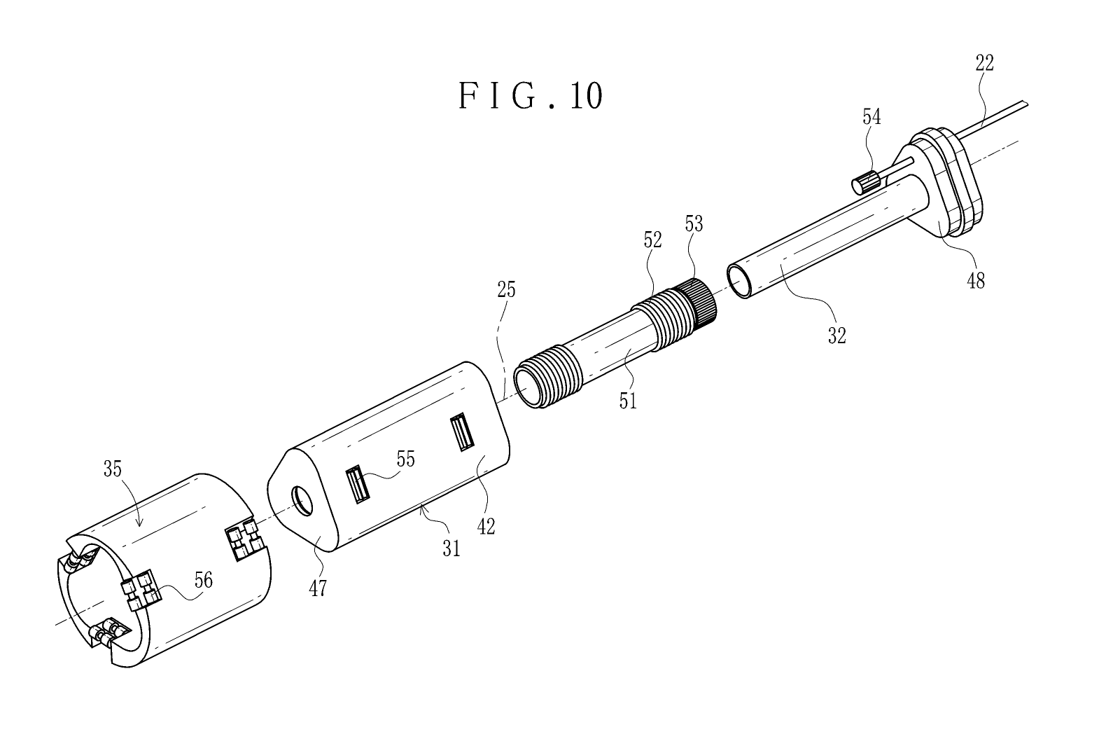

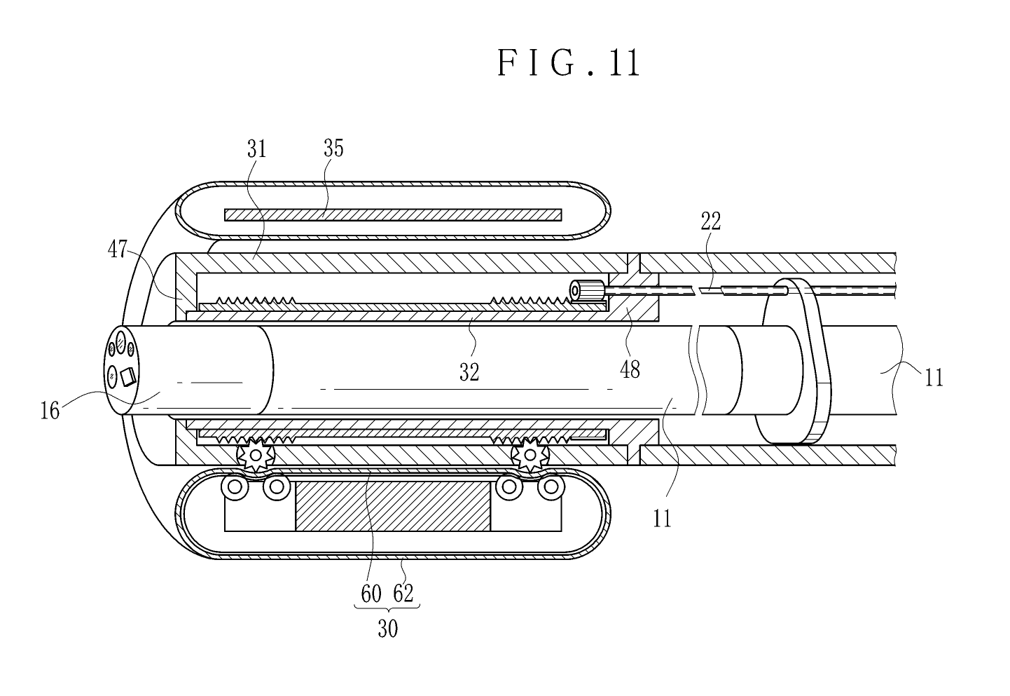

A passage space 38 is defined between the outer sleeve 31 and the roller support sleeve 35 for disposing and moving the track structure 30. There are a front lid portion 47 (end ring) and a rear lid portion 48 (end ring) for coaxially supporting the inner sleeve 32 and the outer sleeve 31. Note that the front lid portion 47 can be formed together with the outer sleeve 31. The rear lid portion 48 can be formed together with the inner sleeve 32 (See FIGS. 10 and 11).

Drive wheels 55 are secured to the three flat portions 42 of the outer sleeve 31 in two positions arranged along the longitudinal axis 25. The drive wheels 55 are so-called worm wheels, and have teeth of a helical gear on its cylindrical surface for mesh with worm gears 52. The drive wheels 55 are so disposed that tip portions of the helical gear protrude from surfaces of the flat portions 42, are meshed with the worm gears 52 inside the flat portions 42, and are meshed with the belt portion 33 of the track structure 30 externally. The drive wheels 55 nip the belt portion 33 in cooperation with the follower rollers 56 disposed on the flat portions 36 of the roller support sleeve 35, and move the belt portion 33 along the longitudinal axis 25 to move the track structure 30 endlessly.

The two follower rollers 56 are supported in a rotatable manner, and are disposed upstream and downstream of the drive wheels 55, to push the belt portion 33 on the drive wheels 55. Each of the follower rollers 56 nips the belt portion 33 in cooperation with the drive wheels 55. The belt portion 33 is curved between one of the drive wheels 55 and two of the follower rollers 56, and moved in the longitudinal direction by the drive wheels 55. The belt portion 33 is always pressed between the drive wheels 55 and the follower rollers 56, but does not have a problem of durability, because formed with high rigidity.

A driver 50 (gear mechanism) is provided in the drive unit 40 for exerting rotational force to the drive wheels 55. The driver 50 includes a gear shaft 51 (drive sleeve) and a drive gear 54 (small gear). The gear shaft 51 is externally supported around the inner sleeve 32 and kept rotatable. The drive gear 54 is supported on the rear lid portion 48 in a rotatable manner. The worm gears 52 are formed on the gear shaft 51 in two positions. A spur gear 53 is formed at one end of the gear shaft 51. The drive gear 54 is disposed between the corner portions 43 of the outer sleeve 31 and the inner sleeve 32, and meshed with the spur gear 53. The torque wire 22, which is penetrated through a wire entry hole 59 formed in the rear lid portion 48, is coupled to the drive gear 54. Rotational force of the torque wire 22 is transmitted by the drive gear 54 to the gear shaft 51.

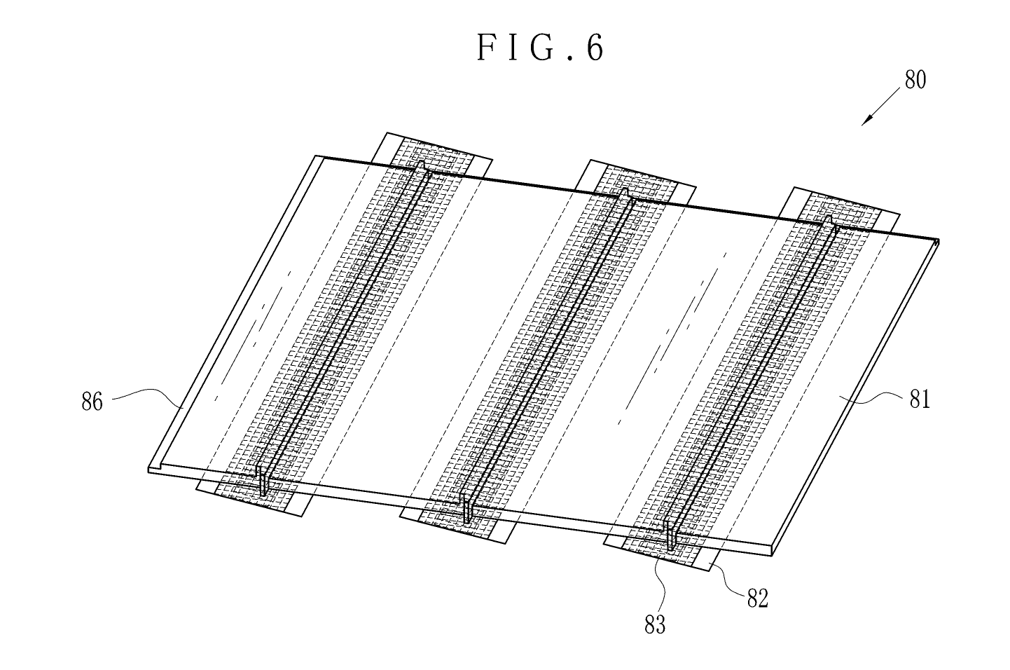

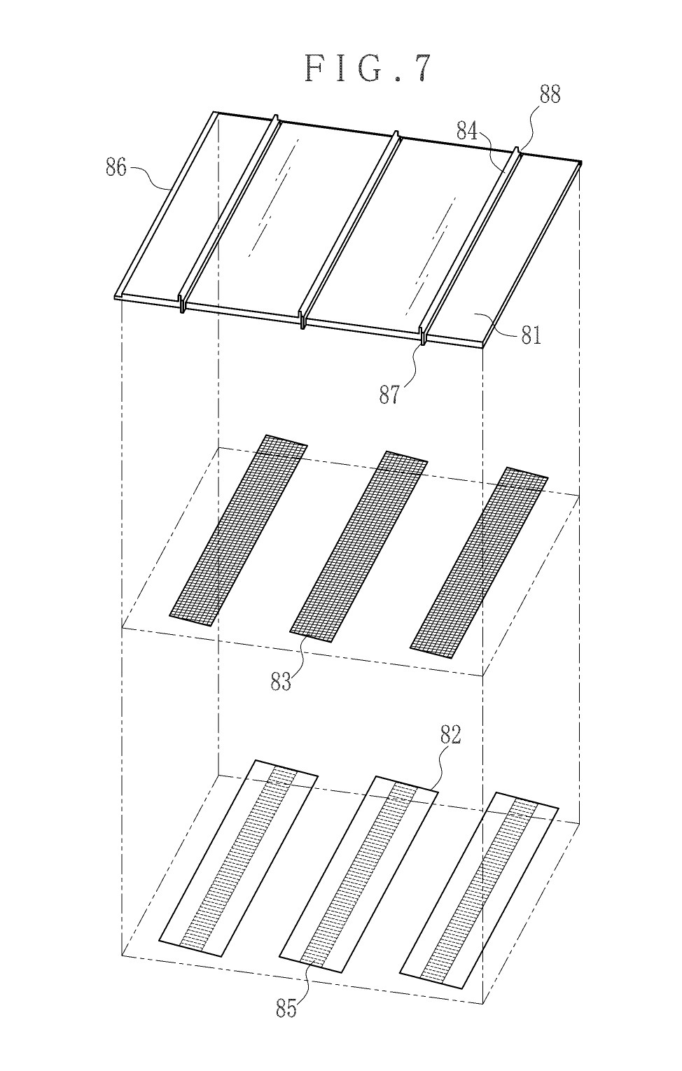

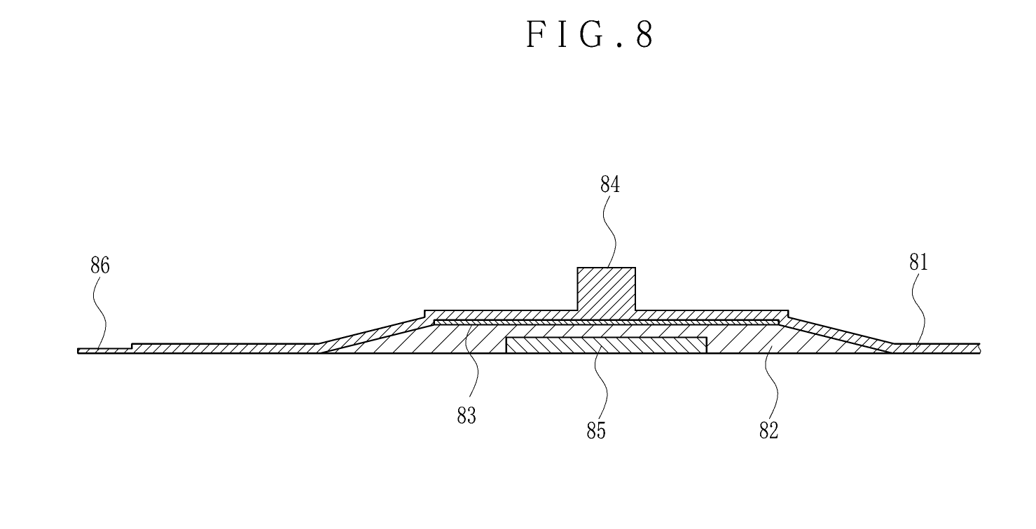

As illustrated in FIGS. 6-8, a material sheet 80 (film sheet material) for forming the track structure 30 is constituted by a polyurethane sheet 81 (sheet layer), three polyurethane sheets 82 (sheet layers) and three nylon mesh sheets 83 (sheet layers). Three ridges 84 are formed on one surface of the polyurethane sheet 81. A rack gear 85 is provided at a center of each of the polyurethane sheets 82. The nylon mesh sheets 83 are sandwiched by the polyurethane sheet 81 and the polyurethane sheets 82 to position the ridges 84 and the rack gear 85 on the inner and outer surfaces of the material sheet 80 for the track structure. Centers of the three polyurethane sheets 82 and the three nylon mesh sheets 83 are aligned with the ridges 84, and attached together by respectively adhesion or thermal welding. Also, a mold set can be used to form the material sheet 80 where the nylon mesh sheets 83 are contained or encapsulated.

The polyurethane sheet 81 is a main sheet, and has flexibility and compressibility/expandability. The nylon mesh sheets 83 are reinforcement sheets, and are attached to the main sheet to constitute the belt portion 33. The belt portion 33 structurally has three layers including the polyurethane sheet 81, the polyurethane sheets 82 and the nylon mesh sheets 83. Note that the each of the main sheet and the reinforcement sheets can be a multi-layer sheet formed by attachment of plural sheet layers. Also, it is possible to form the main sheet with a locally larger thickness to provide the belt portion 33.

The nylon mesh sheets 83 have a very strong property, and have good resistance to abrasion and good durability. The nylon mesh sheets 83 are formed by cutting a nylon mesh ribbon in which a mesh is formed in a lattice pattern in a longitudinal direction. The ridges 84 of the polyurethane sheet 81 are engaged with the groove 57 formed at the center of the follower rollers 56 for endless movement of the track structure 30 straight without a twist. The polyurethane sheet 81 has an adhesive lap portion 86 formed peripherally with a small thickness. Note that each of the various sheet layers in the material sheet 80 can be a sheet of resin with an appropriate function in consideration of the purpose of the endoscope in addition to the above-described examples of the substances.

The rack gear 85 is disposed for mesh with teeth of the helical gear formed with the drive wheels 55, and formed with an inclination of 13.2 degrees equal to that of the helical gear with reference to the longitudinal direction of the polyurethane sheets 82. The surface having the rack gear 85 moves as the working run 62 for contacting an inner wall of the large intestine in the form of the track structure 30. The rack gear 85 is formed so that a tooth end of the rack gear 85 does not protrude from the surface of the polyurethane sheets 82.

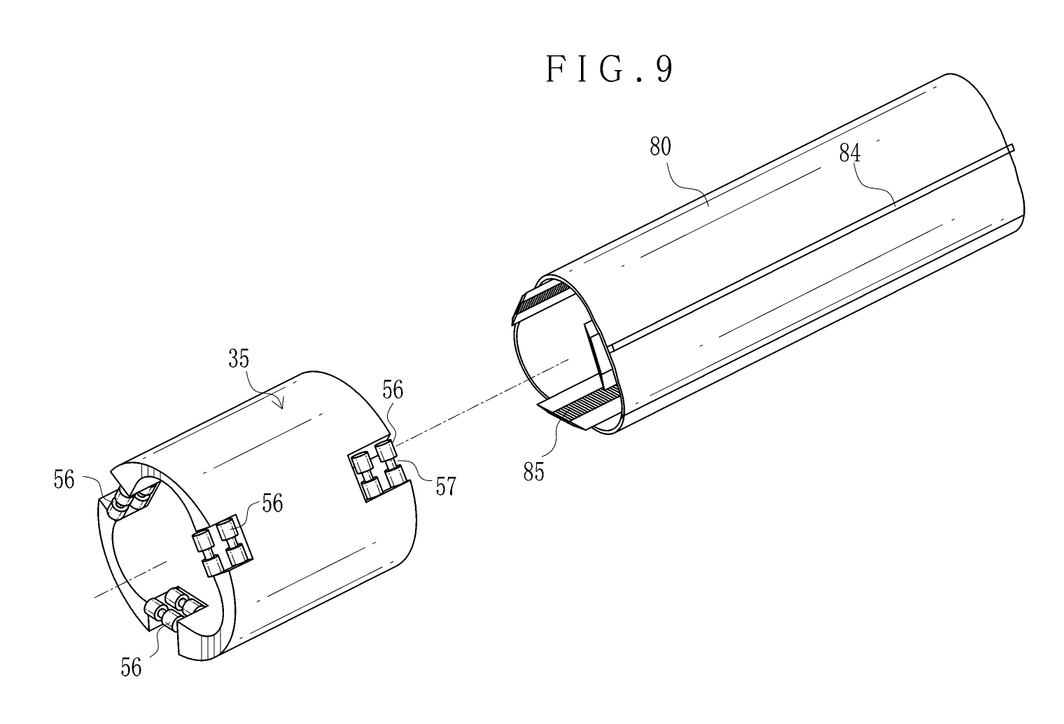

As illustrated in FIG. 9, the material sheet 80 for the track structure is formed tubularly by adhesion of both its edge portions parallel to the ridges 84 of the polyurethane sheet 81 in directing the rack gear 85 internally. The ridges 84 are aligned with the groove 57 formed at the center of the follower rollers 56, while the material sheet 80 in the tubular shape is penetrated through the roller support sleeve 35. Its end portions are bent back at 180 degrees to wrap the roller support sleeve 35, and are attached to one another outside the roller support sleeve 35 to form the bag arrangement of the toroid form. At this time, a projection 87 is fitted in a recess 88 at the ends of the ridges 84.

For example, for the propulsion of the self-propelling device 20 in the large intestine, a rear end of the belt portion 33 is pulled by the drive wheels 55 and the follower rollers 56 disposed in the rear of the drive unit 40 (arrow 26) as illustrated in FIG. 5. A front end of the belt portion 33 is pushed forwards by the drive wheels 55 and the follower rollers 56 disposed in the front of the drive unit 40 (arrow 27). The track structure 30 being pushed becomes spread externally at the front end, is bent back externally by turning at 180 degrees, and contacts the inner wall of the large intestine.

On the other hand, the belt portion 33 on the rear end side in the advancing direction is pulled, so that the working run 62 of the toroid in contact with the inner wall of the gastrointestinal tract is turned over at 180 degrees and bent back internally. Thus, the working run 62 of the track structure 30 is endlessly moved in the return direction (arrow 28), and the return run 60 is endlessly moved in the same direction as the advancing direction (arrow 27), to move the self-propelling device 20 forwards. The tip device 16 of the endoscope 10 is propelled relative to the large intestine. If one wishes to return the self-propelling device 20 relative to the advancing direction, the track structure 30 can be endlessly moved in a direction opposite to the above.

The track structure 30, as its belt portion with rigidity is moved while nipped between the drive wheels 55 and the follower rollers 56, will not twist.

Note that shapes of the sections of the inner surface 45 of the roller support sleeve 35 and the outer surface 41 of the outer sleeve 31 are substantially triangular. However, those shapes may be quadrangular, pentagonal or polygonal in other manners. However, the triangular shapes are still preferable, because an increase in the number of the corners will make the contour near to a circle, to reduce the effect of the deformation.

In the above embodiment, the drive wheels 55 are offset from the follower rollers 56 in the advancing direction. However, at least one of the drive wheels 55 can be opposed directly to one of the follower rollers 56 in relation to the advancing direction. In the above embodiment, the drive wheels 55 are overlapped on the follower rollers 56 in the advancing direction. However, the drive wheels 55 can be disposed in correspondence with a space between the follower rollers 56 without the overlap.

In the above embodiment, an outer surface of the roller support sleeve 35 is cylindrical in contrast with the inner surface 45 in a shape of a triangular prism. However, the outer surface of the roller support sleeve 35 can be formed in a smoothly curved shape other than the cylindrical shape.

For the sheet layers in the material sheet 80, various methods may be used for lamination or forming or those on one another. Combinations of the sheet layers can be determined differently from those of the above-described sheet layers.

Although the present invention has been fully described by way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. A self-propelling device comprising:
a toroid unit, including a track structure shaped in a toroid form with an inner space and movable in circulation, a highly rigid belt portion formed with said track structure to extend in a direction of said circulation, a roller support sleeve, disposed in said inner space of said track structure, and having an inner surface including plural first flat portions and plural first corner portions arranged alternately, and a follower roller, secured to each of said first flat portions, for pressing said belt portion on an inner surface of said track structure;
a drive unit, including an inner sleeve for mounting on an introducer of an endoscope, an outer sleeve having an outer surface including plural second flat portions and plural second corner portions arranged alternately, said plural second flat portions being supported by said inner sleeve, covering an outer surface of said inner sleeve, and being opposed to respectively said first flat portions of said roller support sleeve, said plural second corner portions being opposed to respectively said first corner portions of said roller support sleeve, and a drive wheel for nipping said belt portion with said follower roller of said toroid unit, and moving said track structure in circulation.

2. A self-propelling device as defined in claim 1, wherein said track structure includes at least one main sheet shaped in said toroid form, and said belt portion includes at least one reinforcement sheet attached to said main sheet.

3. A self-propelling device as defined in claim 2, wherein said reinforcement sheet is a mesh sheet of resin.

4. A self-propelling device as defined in claim 1, wherein said first flat portions are three first flat portions, said second flat portions are three second flat portions, said first corner portions are three first corner portions, said second corner portions are three second corner portions, said first and second corner portions are arcuate, and thus an inner surface of said roller support sleeve and an outer surface of said outer sleeve extend substantially triangularly.

5. A self-propelling device as defined in claim 1, wherein a passage space is defined between said roller support sleeve and said outer sleeve for moving said track structure, and each of said follower roller and said drive wheel is disposed partially to project into said passage space.

6. A self-propelling device as defined in claim 1, wherein said drive unit includes front and rear lid portions for coaxially supporting said inner sleeve and said outer sleeve.

7. A self-propelling device as defined in claim 6, wherein said front lid portion is formed together with said outer sleeve.

8. A self-propelling device as defined in claim 6, wherein said rear lid portion is formed together with said inner sleeve.

9. A self-propelling device as defined in claim 1, wherein said drive unit has a driver for applying rotational force to said drive wheel.

10. A self-propelling device as defined in claim 9, wherein said driver includes a worm gear for engagement with said drive wheel, and a drive gear for rotating said worm gear.

11. A self-propelling device as defined in claim 10, wherein said drive gear is disposed inside one of said second corner portions of said outer sleeve.

12. A self-propelling device as defined in claim 10, wherein rotational force is applied to said drive gear with a torque wire by a force source disposed externally of said endoscope.
